(19) United States
(12) Patent Application Publication
(10) Pub. No.: US 2008/0097596 A1
(43) Pub. Date: Apr. 24, 2008

(54) INJECTION OF ANTI-PRESBYOPIA CORRECTIVE ELEMENT PRECURSOR

(76) Inventors: F. Richard Christ, Laguna Beach, CA (US); Marie Dvorak Christ, (US)

Correspondence Address: SHELDON MAK ROSE & ANDERSON PC, 100 East Corson Street, Third Floor, PASADENA, CA, 91103-3842, US

(21) Appl. No.: 11/550,729

(22) Filed: Oct. 18, 2006

(51) Int. Cl. A61F 2/14

(52) U.S. Cl. 623/4.1; 604/506

(57) ABSTRACT

A method for implanting a corrective element into the sclera of an eye for the treatment of presbyopia includes the steps of (a) disposing a precursor of a corrective element at a predetermined position within the sclera; and (b) causing the precursor of a corrective element to form a corrective element in situ.

### FIELD OF THE INVENTION

This invention relates generally to the treatment of presbyopia and, more specifically, to the treatment of presbyopia by the insertion of scleral implant elements.

### BACKGROUND

Presbyopia is a loss or reduction of the accommodating power of the eye which takes place when a person ages. FIG. 1 is a diagrammatic representation of an eye 1 showing the lens 2 enclosed in the lens sac 3 and suspended from the ciliary body 4 by means of the zonule 5. The ciliary body 4 lines the internal surface of the sclera 6 about a ring located on the average at a latitude distance by 2 to 3 mm from the limbus 7, measured along the optical axis.

New methods for treating presbyopia have recently been disclosed wherein implant elements are disposed within small tunnels formed within the sclera of the patient's eyes. Once disposed in the scleral tunnels, the implants act on the sclera to enhance the ability of the patient's lens to contract, thereby diminishing the presbyopia condition. Examples of such new methods are disclosed, for example, in U.S. Pat. Nos. 6,682,560 and 6,692,524, the entireties of which are incorporated herein by this reference.

FIG. 2 illustrates one of these new methods. An corrective element 8 is surgically disposed within an incision tunnel 9 formed in the sclera 6 opposite the zonule 5. The tunnel 9 is disposed at a depth of about 600μ below the surface of the sclera 6. The tunnel 9 is typically about 7 mm long and about 1.5 mm wide. The corrective element 8 favorably effects the adjustment of the lens shape by the sclera 6 and the zonule 5 to minimize the effects of presbyopia.

In such new methods, the implant elements have had to be disposed within the sclera in a two-step process. In a first step, a small tunnel is formed within the sclera of the patient's eye. After the small tunnel is formed, in a second step, the surgeon physically inserts an implant into the tunnel using some form of gripping tool, such as a small forceps. This two-step process of inserting the scleral implant elements is both awkward and time-consuming.

Accordingly, there is a need for a new process of inserting insert elements into the scleral which avoids these problems in the prior art.

### SUMMARY

The invention satisfies this need. The invention is a method for implanting a corrective element into the sclera of an eye. The method comprises the steps of (a) providing a hollow injection needle having a sharpened injection needle tip; (b) piercing the sclera with the injection needle tip and locating the injection needle tip at a predetermined position within the sclera; (c) causing a precursor of a corrective element to move through the injection needle and out of the injection needle tip, so as to be deposited at the predetermined position within the sclera; and (d) causing the precursor of a corrective element to form a corrective element in situ.

### DRAWINGS

FIG. 1 is a schematic view in cross-section of an eye;

FIG. 2 is a fragmentary cross-sectional view of an eye in which is implanted a corrective element for the treatment of presbyopia;

FIG. 3 is a diagrammatic view of a hollow injection needle useful in the method of the invention;

FIG. 4A is a first diagrammatic view of a trocar useful in the method of the invention showing a corrective element retained within its tip;

FIG. 4B is a second diagrammatic view of the trocar illustrated in FIG. 4A, showing the corrective element moved outside the tip of the trocar;

FIG. 4C is a third diagrammatic view of the trocar illustrated in FIG. 4A, showing the corrective element separated from the trocar;

FIGS. 5A-5C illustrate, in diagrammatic form, a tool useful in the method of the invention;

FIG. 5D illustrates in diagrammatic form a corrective element implanted in a sclera using the tool illustrated in FIGS. 5A-5C;

FIGS. 5E-5H illustrate, in diagrammatic form, an alternative tool useful in the method of the invention;

FIGS. 6A-6C illustrate different embodiments of the tool illustrated in FIGS. 5E-5H, each embodiment having a different handle configuration;

FIG. 7A is a cross-sectional side view of another embodiment of a tool useful in the method of the invention, showing the tip of the tool in a retracted position;

FIG. 7B is a cross-sectional side view of the tool embodiment illustrated in FIG. 7A, showing the tip of the tool in an extended position; and

FIG. 7C is a cross-sectional side view of the use of the tool illustrated in FIG. 7A showing its use in the injection of a corrective element into the precursor of a corrective element.

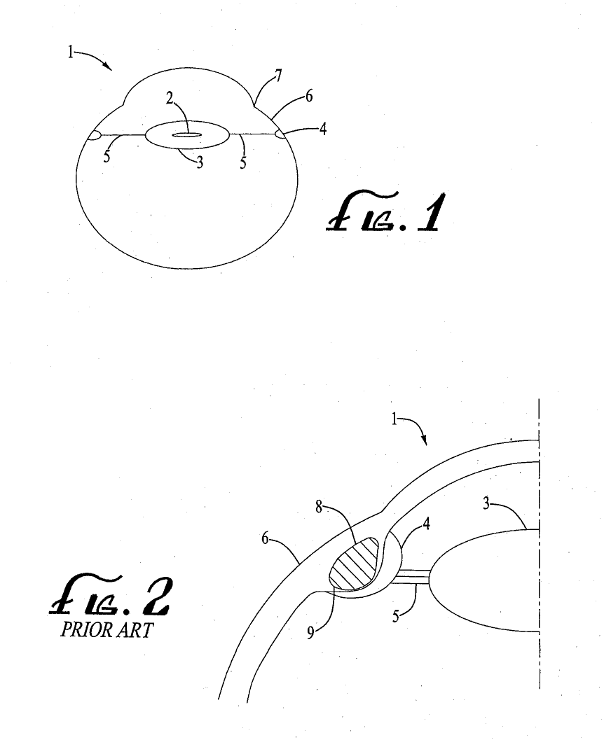

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

The invention is a method for implanting a precursor 11 of a corrective element 8 into the sclera 6 of an eye 1 for the treatment of presbyopia. The method comprises the steps of (a) providing a hollow injection needle having a sharpened injection needle tip; (b) piercing the sclera with the injection needle tip and locating the injection needle tip at a predetermined position within the sclera; (c) causing a precursor 11 of a corrective element 8 to move through the injection needle and out of the injection needle tip, so as to be deposited at the predetermined position within the sclera; and (d) causing the precursor 11 of a corrective element 8 to form a corrective element in situ.

FIG. 3 illustrates in diagrammatic form a hollow injection needle 10 wherein there is disposed a plurality of precursors 11 of corrective elements 8. This injection needle 10 is a syringe. The injection needle 10 has a sharpened injection needle tip 12 and an oppositely disposed plunger 18. The plunger 18 can be used to cause the precursors 11 of corrective elements 8 to move through the injection needle 10 and out of the injection needle tip 12.

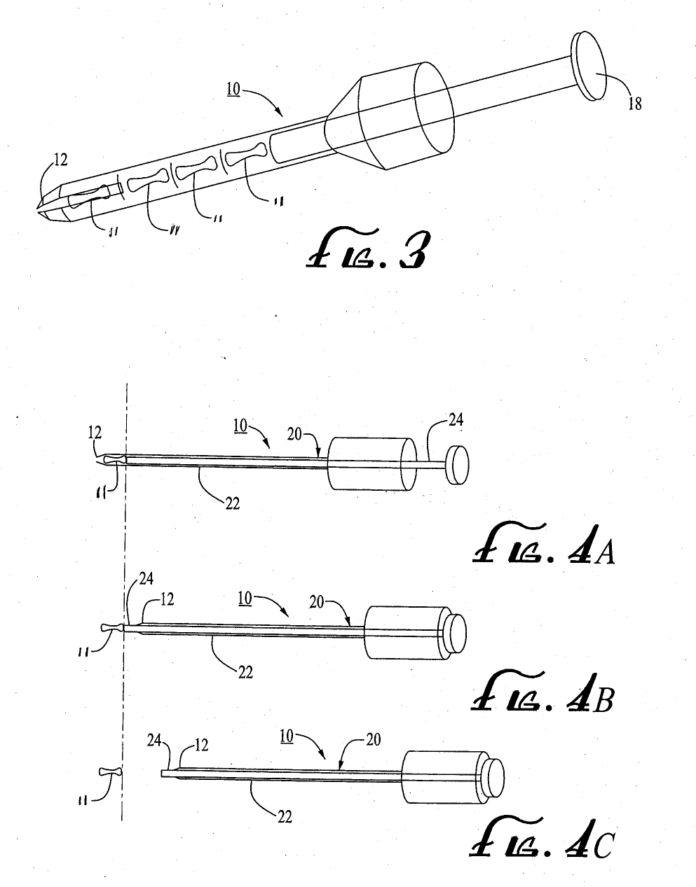

FIGS. 4A, 4B and 4C illustrate an alternative hollow injection needle 10. The hollow injection needle 10 illustrated in FIGS. 4A, 4B and 4C is a trocar 20 having a barrel 22 with a sharpened injection needle tip 12 and a slideable, co-axially disposed internal rod 24. The rod 24 is longer than the barrel 22. With this embodiment, a precursor 11 of a corrective element 8 can be pre-loaded into the injection needle tip 12 (as illustrated in FIG. 4A). After the injection needle tip 12 is used to pierce the sclera 6 and the injection needle tip 12 is located at the predetermined position 14 within the sclera 6, the internal rod 24 is held steady while the barrel 22 is withdrawn (as illustrated in FIG. 4B). The action of withdrawing the barrel 22 causes the precursor 11 of a corrective element 8 to be separated from the trocar 20 (as illustrated in FIG. 4C).

A precursor 11 of a corrective element 8 can be made by a memory form material, i.e., a material that takes on the shape of a corrective element 8 after it has been implanted into the sclera 5.

Silicone elastomers are one example of such memory form materials. Silicone elastomers are highly deformable so that they can be molded into a desired shape and then stretched by mechanical force to provide a thinner, more elongate shape suitable for injection. After mechanical force is released, the silicone elastomer immediately relaxes to its original shape. Silicone elastomers can be made in a range of hardnesses. Also, silicone elastomers can also be readily stretched within an injection needle 10. Silicone elastomeric devices can be fabricated by molding and therefore have the potential to be manufactured at lower costs than PMMA devices which must be machined. Also, silicone can be sterilized by autoclave steam whereas PMMA requires ethylene oxide gas.

Another example of memory form materials are shape memory materials. Nitinol materials is one example of such shape memory materials. Also, shape memory polymers are known in the art which can be formed at a first temperature, deformed at a cooler temperature and then returned to its original shape when returned to the first temperature. Such shape memory polymers are disclosed, for example, in U.S. Pat. No. 6,679,605, the contents of which are incorporated herein in its entirety by this reference.

Shape memory materials are typically introduced in an elongated rod format. The implant re-assumes its original as molded shorter, fatter shape upon equilibration to eye temperature.

Yet another example of memory form materials are hydrogel polymers known in the art and capable of taking on a predetermined shape once they are hydrated. Precursors 11 of corrective elements 8 made from such hydrogel polymers are injected into the scleral in a small, dry configuration. Once in the sclera, the polymers are hydrated by moisture within the sclera and are thereby caused to expand to the predetermined shape of the corrective element 8.

Memory form materials can be introduced into the sclera using an injection needle 10 such as that which is illustrated in FIG. 3 or a trocar 20 such as that which is illustrated in FIGS. 4A-4C.

An alternative precursor 11 of a corrective element 8 comprises a fillable balloon having a predetermined inflation shape. The balloon can be made of polyethylene terephthalate (PET) polymer which will not expand on its “as formed” shape and size. Alternatively, the balloon can be made of silicone or polyurethane polymers which can stretch and can therefore be expanded beyond its “as formed” shape and size.

The balloon is inflated with an in situ curable polymer material (either thermally or photochemically cured). Examples are platinum-catalyzed silicone rubber or UV-light initiated acrylates including PMMA or soft acrylates. The concept is similar to balloon catheters used in cardiovascular and other fields. An example of such technology is taught in U.S. Pat. No. 5,769,817, the contents of which are incorporated in its entirety herein by this reference.

In a preferred embodiment of the method of the invention, the method comprises the additional step of, prior to piercing the sclera 6 in step (b), the outer scleral surface 38 adjacent to the predetermined position 14 within the sclera 6 is flattened. It is desirable to flatten the sclera 6 so as to create a channel 26 in the sclera 6 which is parallel with the scleral surface 38. Flattening the sclera 6 facilitates this and reduces the risk of perforating the inner scleral surface.

Such flattening can be accomplished using equipment and techniques described in U.S. Pat. Nos. 5,556,406, 5,586,980 and 6,083,236, the entireties of which are incorporated herein by these references. Each of these patents describe a circular ring that has roughly the diameter of a cornea. For use in the present invention, a more limited vacuum positioning device that flattens just the sclera 6 immediately adjacent to the side of the tunnel incision is used. Thus, the device is smaller and alternatively rectangular in shape with a long dimension in the same direction as the incision.

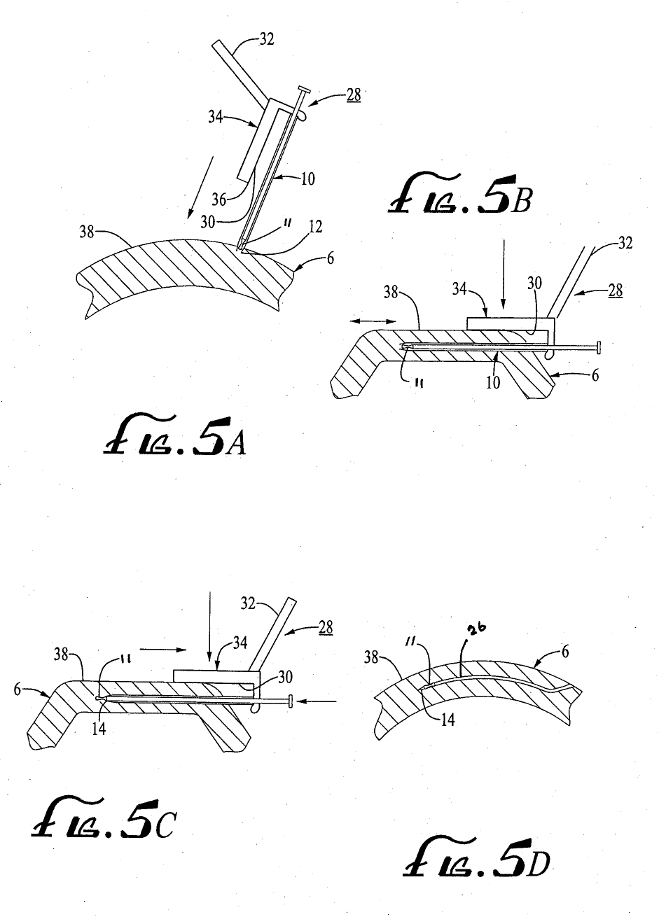

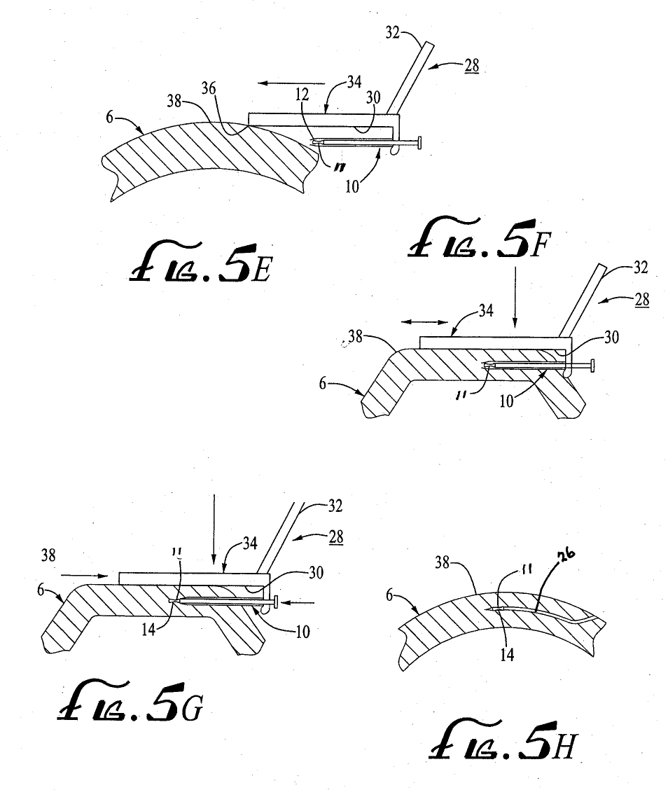

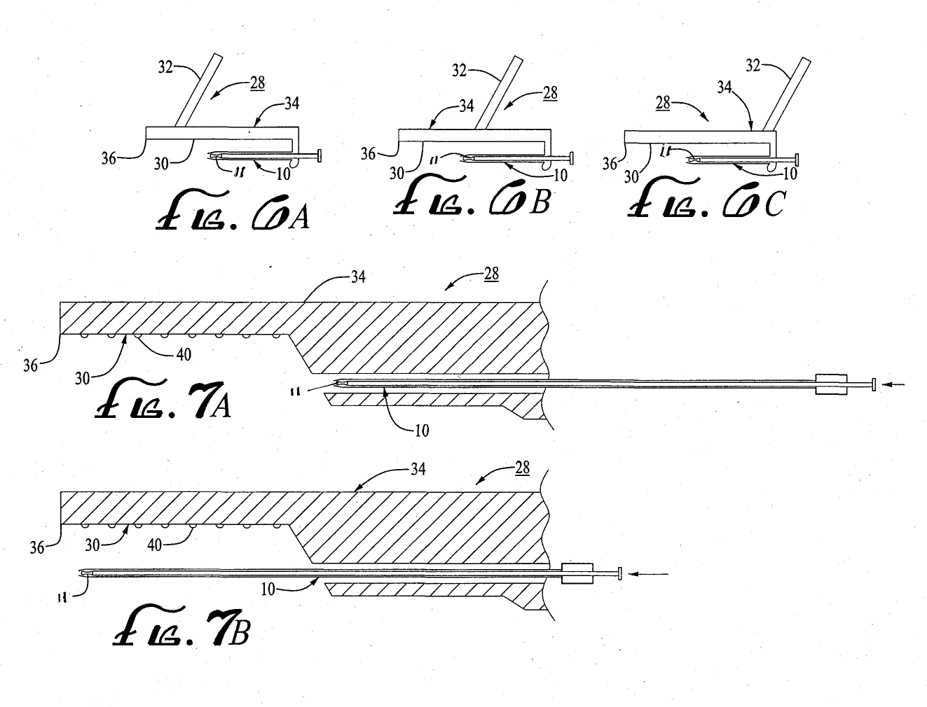

Alternatively, such flattening can be accomplished using a combination 28 comprising an injection needle 10 and scleral flattening surface 30. FIGS. 5A-5B illustrate diagrammatically such a combination. The combination illustrated in FIGS. 5A-5B comprise a handle 32, a retaining element 34, a trocar 20, such as illustrated in FIGS. 4A-4B, and a scleral flattening surface 30 having a scleral flattening surface tip 36. In the combination illustrated in FIGS. 5A-5B, the injection needle tip 12 extends outwardly a distance between about 0.5 mm and about 2 mm, and typically between about 1 mm and about 2 mm, beyond the scleral flattening surface tip 36.

The combination illustrated in FIGS. 5A-5B can be used to inject a precursor 11 of a corrective element 8 into the sclera 6 of the eye 1 by the following steps. First, the injection needle 10 is disposed approximately orthogonally to the outer surface 38 of the sclera 6 as illustrated in FIG. 5A. While the injection needle 10 is disposed orthogonally to the surface 38 of the sclera 6, the injection needle tip 12 is contacted with the outer surface 38 of the sclera 6 and the injection needle tip 12 is used to penetrate the sclera 6. Next, the scleral flattening surface 30 is used to apply pressure to a portion of the surface 38 of the sclera 6 to flatten that portion of the scleral surface 38, and the injection needle 10 is rotated so that the injection needle 10 is disposed parallel to the flattened surface portion 38 of the sclera 6. Next, the injection needle 10 is moved parallel to the flattened portion of the sclera 6 to continue the insertion of the injection needle 10 into the sclera 6 until the injection needle tip 12 is disposed at the predetermined position 14 within the sclera 6 (as illustrated in FIGS. 5B and 5C). When the injection needle tip 12 is disposed at the predetermined position 14, the precursor 11 of a corrective element 8 is moved out of the injection needle 10 and into the sclera 6. Lastly, the injection needle 10 is retracted from the sclera 6 and the scleral flattening surface 30 is removed from contact with the outer surface 38 of the sclera 6, leaving behind the precursor 11 of a corrective element 8 at the predetermined position 14 within the sclera 6. When this is done, the surface 38 of the sclera 6 returns to its natural arcuate shape (as illustrated in FIG. 5D).

FIGS. 5E-5H illustrate another embodiment of a combination of an injection needle 10 and a scleral flattening surface 30. The embodiment illustrated in FIGS. 5E-5H is similar to the design of the embodiment illustrated in FIGS. 5A-5B, except that the scleral flattening surface tip 36 extends outwardly a distance greater than the injection needle tip 12. The use of the combination illustrated in FIGS. 5E-5H is described as follows. As illustrated in FIG. 5E, the scleral flattening surface 30 is first used to press down on a portion of the surface 38 of the sclera 6 to flatten it. Next, the injection needle 10 is moved laterally, parallel with the flattened surface 30 of the sclera 6, so that the injection needle 10 is inserted into the sclera 6 generally parallel to the flattened surface 30 of the sclera 6 until the injection needle tip 12 is disposed at the predetermined position 14 within the sclera 6 (as illustrated in FIGS. 5F and 5G). Once the injection needle tip 12 is disposed at the predetermined position 14 within the sclera 6, the precursor 11 of a corrective element 8 is moved out of the injection needle 10 and into the sclera 6. Finally, the injection needle 10 is retracted from the sclera 6 and the sclera flattening surface 30 is removed from contact with the surface 38 of the sclera 6. When this is accomplished, the surface 38 of the sclera 6 returns to its natural arcuate shape as illustrated in FIG. 5H.

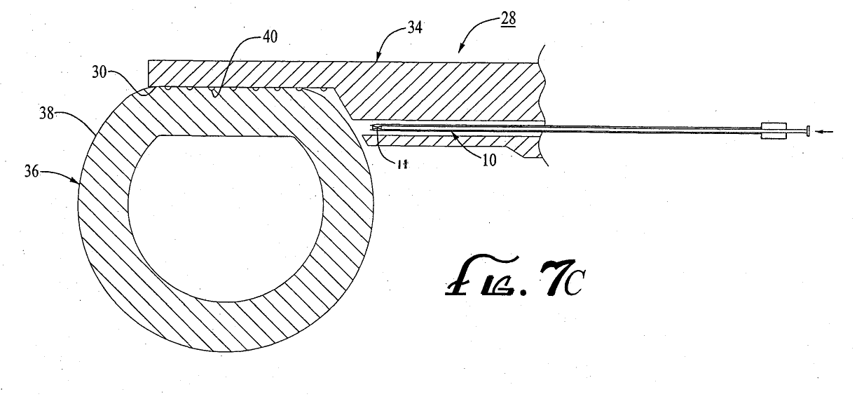

FIGS. 6A-6C illustrate three different additional embodiments of the combination 28 comprising an injection needle 10 and scleral flattening surface 30. Each of these three embodiments differs from one another by the placement of the handle 32.

FIGS. 7A and 7B illustrate yet another combination 28 comprising a slideable needle 10 and scleral flattening surface 30. In this embodiment, the injection needle 10 is slidably retained within the retaining element 34 and is capable of alternatively moving between an extended position (illustrated in FIG. 7B) and a retracted position (illustrated in FIG. 7A). In this embodiment, it is preferred that the scleral flattening surface 30, although being generally smooth, defines tiny traction elements 40 for minimizing slippage of the scleral flattening surface 30 along the surface 38 of the sclera 6. The length of the scleral flattening surface 30 in this embodiment is typically about 10 mm.

FIG. 7C illustrates the use of the embodiment shown in FIGS. 7A and 7B to insert a precursor 11 of a corrective element 8 within the sclera 6. The scleral flattening surface 30 is placed in contact with the surface 38 of the sclera 6 and pressed down to flatten that portion of the scleral surface 38. Next, the injection needle 10 is caused to move from its retracted position to its extended position. As this is accomplished, the injection needle 10 pierces the sclera 6 in a direction which is generally parallel to the flattened portion of the scleral surface 38. Once the injection needle tip 12 is disposed at the predetermined position 14, the precursor 11 of a corrective element 8 is moved out of the injection needle 10 and into the sclera 6. Thereafter, the injection needle 10 is moved to the retracted position, the scleral flattening surface 30 is disengaged from the surface 38 of the sclera 6 and the surface 38 of the sclera 6 returns to its natural curvature.

The invention offers many advantages over prior art methods of implanting pre-formed corrective elements into the sclera. First and foremost, the invention simplifies the surgical procedure by accomplishing the implant in a single step, rather in two steps as required by the prior art. The one-step procedure of the invention simplifies and shortens the surgical procedure, thereby reducing the expense to the patient and thereby making it easier for the surgeon to successfully accomplish the surgery. The one-step procedure also minimizes the chances of errors arising from the surgical procedure, because the channel within the sclera is formed and the corrective element is disposed within that channel in a single step. Errors arising from the second step in prior art methods (i.e., errors arising from the improper disposition of a preformed corrective element into a properly located channel) are eliminated.

An additional advantage of the invention arises from the fact that most corrective element precursors can be made to more fully “fill up” the insertion channel. This minimizes problems caused by preformed corrective elements migrating or rotating within the implant channel.

Also, most corrective element precursors can be caused to swell within the implant channel, thereby overfilling the channel. This further minimizes the chances of the corrective element migrating or rotating within the channel. Moreover, for the same diameter channel, corrective elements implanted by the invention can exert additional pressure against the ciliary muscle over the pressure exerted by the implantation of a preformed corrective element.

The implantation of corrective element precursors also minimizes damage caused to the sclera by having to forcibly slide the preformed corrective elements through the implant channel.

The use of corrective element precursors also provides the ability to implant corrective elements of complex shape, shapes which in a preformed element would be difficult to move through the implant channel without causing damage to the sclera tissue.

Still further, many of the corrective elements formed in the invention can be formed of a softer material than those used in the prior art. Softer materials are less likely to cause damage and subsequent inflammation to the sclera tissue and provide additional traction within the implant channel so as to minimize migration and rotation of the corrective element within the channel.

Finally, in the case of corrective element precursors using inflatable balloons, the pressure exerted by the corrective element on the sclera can be finely tuned by adjusting the inflation of the balloon.

Accordingly, the invention provides many significant advantages over the prior art.

Having thus described the invention, it should be apparent that numerous structural modifications and adaptations may be resorted to without departing from the scope and fair meaning of the instant invention as set forth hereinabove.

What is claimed is:

1. A method for implanting a corrective element into the scleral of an eye, the method comprising the steps of:
(a) disposing a precursor of a corrective element at a predetermined position within the sclera; and
(b) causing the precursor of a corrective element to form a corrective element in situ.

2. The method of claim 1 wherein the step of disposing the precursor of a corrective element in step (a) is accomplished with a trocar having a slidable, axially disposed internal rod.

3. The method of claim 1 wherein the precursor of a corrective element comprises a memory form material.

4. The method of claim 3 wherein the memory form material comprises silicone elastomer.

5. The method of claim 3 wherein the memory form material comprises a shape memory material.

6. The method of claim 3 wherein the memory form material comprises a hydrogel material.

7. The method of claim 1 wherein the precursor of a corrective element comprises a curable polymer disposed within a balloon having a predetermined as-formed shape.

8. The method of claim 7 wherein the balloon can be expanded beyond its as-formed shape.

9. A method for implanting a corrective element into the sclera of an eye, the method comprising the steps of:
(a) providing a hollow injection needle having a sharpened injection needle tip;
(b) piercing the sclera with the injection needle tip and locating the injection needle tip at a predetermined position within the sclera;
(c) causing a precursor of a corrective element to move through the injection needle and out of the injection needle tip, so as to be deposited at the predetermined position within the sclera; and
(d) causing the precursor of a corrective element to form a corrective element in situ.

10. The method of claim 9 wherein the hollow injection needle comprises a trocar having a slidable axially disposed internal rod.

11. The method of claim 9 wherein the precursor of a corrective element comprises a memory form material.

12. The method of claim 11 wherein the memory form material comprises silicone elastomer.

13. The method of claim 11 wherein the memory form material comprises a shape memory material.

14. The method of claim 11 wherein the memory form material comprises a hydrogel material.

15. The method of claim 9 wherein the precursor of a corrective element comprises a curable polymer disposed within a balloon having a predetermined as-formed shape.

16. The method of claim 15 wherein the balloon can be expanded beyond its as-formed shape.
